(19) United States
(12) Patent Application Publication
(10) Pub. No.: US 2019/0142302 A1
(43) Pub. Date: May 16, 2019

(54) SENSOR DEVICES AND METHODS FOR CONTROLLING A SENSOR DEVICE

(71) Applicant: RAZER (ASIA-PACIFIC) PTE. LTD., Singapore (SG)

(72) Inventors: Sze Wei Joel HONG, Singapore (SG); Jian Yao LIEN, Singapore (SG)

(21) Appl. No.: 16/308,759

(22) PCT Filed: Jun. 16, 2016
(86) PCT No.: PCT/SG2016/050276

§ 371 Date: Dec. 10, 2018

(51) Int. Cl. A61B 5/087; G01N 33/497; H02K 7/18; A61M 16/06

(52) U.S. Cl. CPC A61B 5/0878; A61M 16/06; H02K 7/18; G01N 33/497

(57) ABSTRACT

According to various embodiments, a sensor device may be provided. The sensor device may include: an airflow sensor configured to generate a voltage based on an airflow; and an output circuit configured to output measurement data based on the voltage; wherein the airflow sensor is configured to provide the voltage as a power supply to the output circuit.

### TECHNICAL FIELD

Various embodiments generally relate to sensor devices and methods for controlling a sensor device.

### BACKGROUND

Instruments which measure breathing volumes of performance athletes are usually isolated to labs or require specialized equipment which may be cumbersome and expensive. Measurements are usually performed in a laboratory or a treadmill, which does not simulate real outdoor conditions. Breathing volume measurements are therefore not easily available to sports and fitness enthusiasts. Thus, there may be a need for improved breath measurements.

### SUMMARY OF THE INVENTION

According to various embodiments, a sensor device may be provided. The sensor device may include: an airflow sensor configured to generate a voltage based on an airflow; and an output circuit configured to output measurement data based on the voltage; wherein the airflow sensor is configured to provide the voltage as a power supply to the output circuit.

According to various embodiments, a method for controlling a sensor device may be provided. The method may include: controlling an airflow sensor to generate a voltage based on an airflow; controlling an output circuit to output measurement data based on the voltage; and controlling the airflow sensor to provide the voltage as a power supply to the output circuit.

### BRIEF DESCRIPTION OF THE DRAWINGS

In the drawings, like reference characters generally refer to the same parts throughout the different views. The drawings are not necessarily to scale, emphasis instead generally being placed upon illustrating the principles of the invention. The dimensions of the various features or elements may be arbitrarily expanded or reduced for clarity. In the following description, various embodiments of the invention are described with reference to the following drawings, in which:

FIG. 1A shows a sensor device according to various embodiments;

FIG. 1B shows a sensor device according to various embodiments;

FIG. 1C shows a flow diagram illustrating a method for controlling a sensor device according to various embodiments; and

FIG. 2 shows a functional block diagram of a breath measuring device according to various embodiments.

### DETAILED DESCRIPTION

The following detailed description refers to the accompanying drawings that show, by way of illustration, specific details and embodiments in which the invention may be practiced. These embodiments are described in sufficient detail to enable those skilled in the art to practice the invention. Other embodiments may be utilized and structural, and logical changes may be made without departing from the scope of the invention. The various embodiments are not necessarily mutually exclusive, as some embodiments can be combined with one or more other embodiments to form new embodiments.

In this context, the sensor device as described in this description may include a memory which is for example used in the processing carried out in the sensor device. A memory used in the embodiments may be a volatile memory, for example a DRAM (Dynamic Random Access Memory) or a non-volatile memory, for example a PROM (Programmable Read Only Memory), an EPROM (Erasable PROM), EEPROM (Electrically Erasable PROM), or a flash memory, e.g., a floating gate memory, a charge trapping memory, an MRAM (Magnetoresistive Random Access Memory) or a PCRAM (Phase Change Random Access Memory).

In an embodiment, a “circuit” may be understood as any kind of a logic implementing entity, which may be special purpose circuitry or a processor executing software stored in a memory, firmware, or any combination thereof. Thus, in an embodiment, a “circuit” may be a hard-wired logic circuit or a programmable logic circuit such as a programmable processor, e.g. a microprocessor (e.g. a Complex Instruction Set Computer (CISC) processor or a Reduced Instruction Set Computer (RISC) processor). A “circuit” may also be a processor executing software, e.g. any kind of computer program, e.g. a computer program using a virtual machine code such as e.g. Java. Any other kind of implementation of the respective functions which will be described in more detail below may also be understood as a “circuit” in accordance with an alternative embodiment.

In the specification the term “comprising” shall be understood to have a broad meaning similar to the term “including” and will be understood to imply the inclusion of a stated integer or step or group of integers or steps but not the exclusion of any other integer or step or group of integers or steps. This definition also applies to variations on the term “comprising” such as “comprise” and “comprises”.

The reference to any prior art in this specification is not, and should not be taken as an acknowledgement or any form of suggestion that the referenced prior art forms part of the common general knowledge in Australia (or any other country).

In order that the invention may be readily understood and put into practical effect, particular embodiments will now be described by way of examples and not limitations, and with reference to the figures.

Various embodiments are provided for devices, and various embodiments are provided for methods. It will be understood that basic properties of the devices also hold for the methods and vice versa. Therefore, for sake of brevity, duplicate description of such properties may be omitted.

It will be understood that any property described herein for a specific device may also hold for any device described herein. It will be understood that any property described herein for a specific method may also hold for any method described herein. Furthermore, it will be understood that for any device or method described herein, not necessarily all the components or steps described must be enclosed in the device or method, but only some (but not all) components or steps may be enclosed.

The term “coupled” (or “connected”) herein may be understood as electrically coupled or as mechanically coupled, for example attached or fixed, or just in contact without any fixation, and it will be understood that both direct coupling or indirect coupling (in other words: coupling without direct contact) may be provided.

Instruments which measure breathing volumes of performance athletes are usually isolated to labs or require specialized equipment which may be cumbersome and expensive. Measurements are usually performed in a laboratory or a treadmill, which does not simulate real outdoor conditions. Breathing volume measurements are therefore not easily available to sports and fitness enthusiasts.

To address the above problems, according to various embodiments, a breath intake measurement device may be provided which aims to provide accessibility to fitness enthusiasts to wear on their training. It may also be used for pro-athletes/coaches who are looking to chart their players' progress and statistics on their breathing patterns throughout an exercise.

According to various embodiments, a breath intake measuring device (for example a smart portable fitness breath analyzer) may be provided.

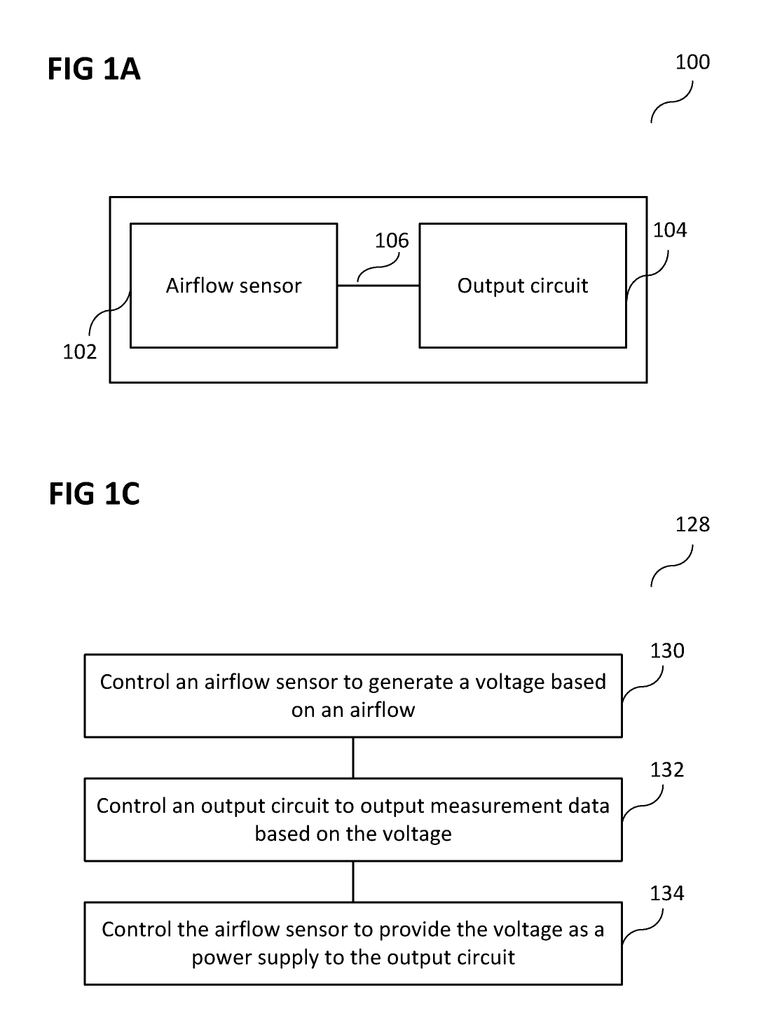

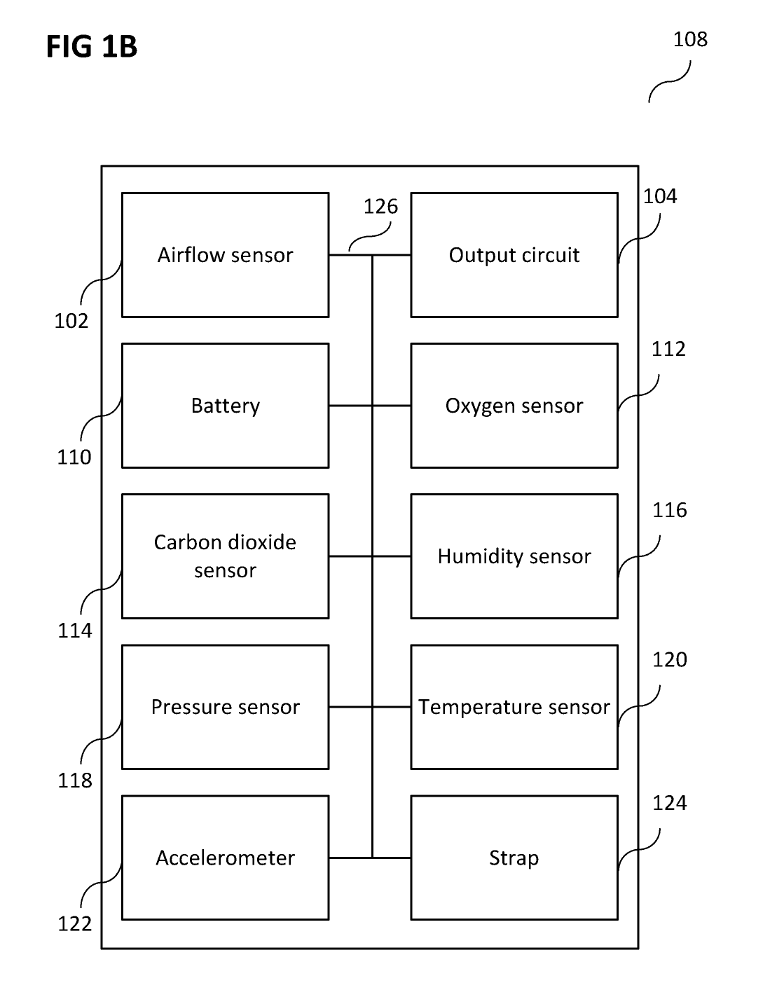

FIG. 1A shows a sensor device 100 according to various embodiments. The sensor device 100 may include an airflow sensor 102 configured to generate a voltage based on an airflow. The sensor device 100 may further include an output circuit 104 configured to output measurement data based on the voltage. The airflow sensor 102 may be configured to provide the voltage as a power supply to the output circuit 104. The airflow sensor 102 and the output circuit 104 may be coupled with each other, like indicated by line 106, for example electrically coupled, for example using a line or a cable, and/or mechanically coupled.

In other words, a sensor device may include an airflow sensor, which may generate a voltage, which may be used as a measurement signal and as power supply.

FIG. 1B shows a sensor device 108 according to various embodiments. The sensor device 108 may, similar to the sensor device 100 of FIG. 1A, include an airflow sensor 102 configured to generate a voltage based on an airflow. The sensor device 108 may, similar to the sensor device 100 of FIG. 1A, further include an output circuit 104 configured to output measurement data based on the voltage. The sensor device 108 may further include battery 110, like will be described below. The sensor device 108 may further include an oxygen sensor 112. The sensor device 108 may further include a carbon dioxide sensor 114. The sensor device 108 may further include a humidity sensor 116. The sensor device 108 may further include a pressure sensor 118. The sensor device 108 may further include a temperature sensor 120. The sensor device 108 may further include an accelerometer 122. The sensor device 108 may further include a strap 124, like will be described below. The airflow sensor 102 may be configured to provide the voltage as a power supply to the output circuit 104. The airflow sensor 102, the output circuit 104, the battery 110, the oxygen sensor 112, the carbon dioxide sensor 114, the humidity sensor 116, the pressure sensor 118, the temperature sensor 120, the accelerometer 122, and the strap 124 may be coupled with each other, like indicated by lines 126, for example electrically coupled, for example using a line or a cable, and/or mechanically coupled.

According to various embodiments, the battery 110 may be configured to power the airflow sensor 102 and/or the output circuit 104.

According to various embodiments, the airflow sensor 102 may be configured to provide the voltage for charging the battery 110.

According to various embodiments, the airflow may be breath.

According to various embodiments, the airflow sensor 102 may include or may be at least one of a turbine or a microelectromechanical system.

According to various embodiments, the output circuit 104 may include or may be an interface.

According to various embodiments, the interface may be configured according to at least one of Bluetooth, Bluetooth low energy, and universal serial bus.

According to various embodiments, the sensor device 108 may be a sensor mask.

According to various embodiments, the strap 124 may be configured to attach the sensor device 108 to a user's head or a user's face.

FIG. 1C shows a flow diagram 128 illustrating a method for controlling a sensor device according to various embodiments. In 130, an airflow sensor may be controlled to generate a voltage based on an airflow. In 132, an output circuit may be controlled to output measurement data based on the voltage. In 134, the airflow sensor may be controlled to provide the voltage as a power supply to the output circuit.

According to various embodiments, the method may further include controlling a battery configured to power the airflow sensor and the output circuit.

According to various embodiments, the airflow sensor may be controlled to provide the voltage for charging the battery.

According to various embodiments, the airflow may be breath.

According to various embodiments, the airflow sensor may include or may be at least one of a turbine or a microelectromechanical system.

According to various embodiments, the method may further include controlling an oxygen sensor.

According to various embodiments, the method may further include controlling a carbon dioxide sensor.

According to various embodiments, the method may further include controlling a humidity sensor.

According to various embodiments, the method may further include controlling a pressure sensor.

According to various embodiments, the method may further include controlling a temperature sensor.

According to various embodiments, the method may further include controlling an accelerometer.

According to various embodiments, the output circuit may include or may be an interface.

According to various embodiments, the interface may be configured according to at least one of Bluetooth, Bluetooth low energy, and universal serial bus.

According to various embodiments, the sensor device may be a sensor mask.

According to various embodiments, the sensor device may include a strap configured to attach the sensor device to a user's head.

According to various embodiments, a breath intake measurement device may be provided which may be lightweight and which does not restrict breathability of the user while simulating real outdoor conditions.

According to various embodiments, the breath intake measurement device may be used during physical activity for the measurement of volume of breath intake.

According to various embodiments, a device (for example a mask) may be provided which covers at least a part of the user's mouth and/or nose, and which may include: A controller unit; a sensor; a turbine for movement of fan blades within the mask (which may give an estimate of air flow, thus giving volume of air inhaled and exhaled, and wherein movement of the turbine may also drive an optional dynamo/battery which may provide power to the electrical components of the mask); and a transmitter for transmitting data to a wearable device, remote server or to a mobile computing device, wherein the data is translated into usable statistics for the wearer's review.

According to various embodiments, a lightweight half-face mask may be provided which covers the mouth and nose of the user. Mask straps at the back of the head may be provided for support like a half-face respirator.

According to various embodiments, large sized air vents may be located at the front of the mask with built in silicone membrane valves for minimization of impediment to air flow. The valves may ensure that air flow only happens in one direction to prevent air-rebreathing and heat build up. This may also improve the accuracy of the sensors measurements of incoming air.

According to various embodiments, a micro MEMS (microelectromechanical system) air flow sensor or a micro wind turbine may be used to measure air flow. Besides measuring air flow, the micro wind turbine may generate energy to self power the fitness breath analyzer.

A BLE (Bluetooth Low Energy) connection may allow the collated data to be easily and wirelessly uploaded to a mobile phone's app (application). A USB (universal serial bus) connection may allow data to be uploaded to any connected PC (personal computer) or laptop.

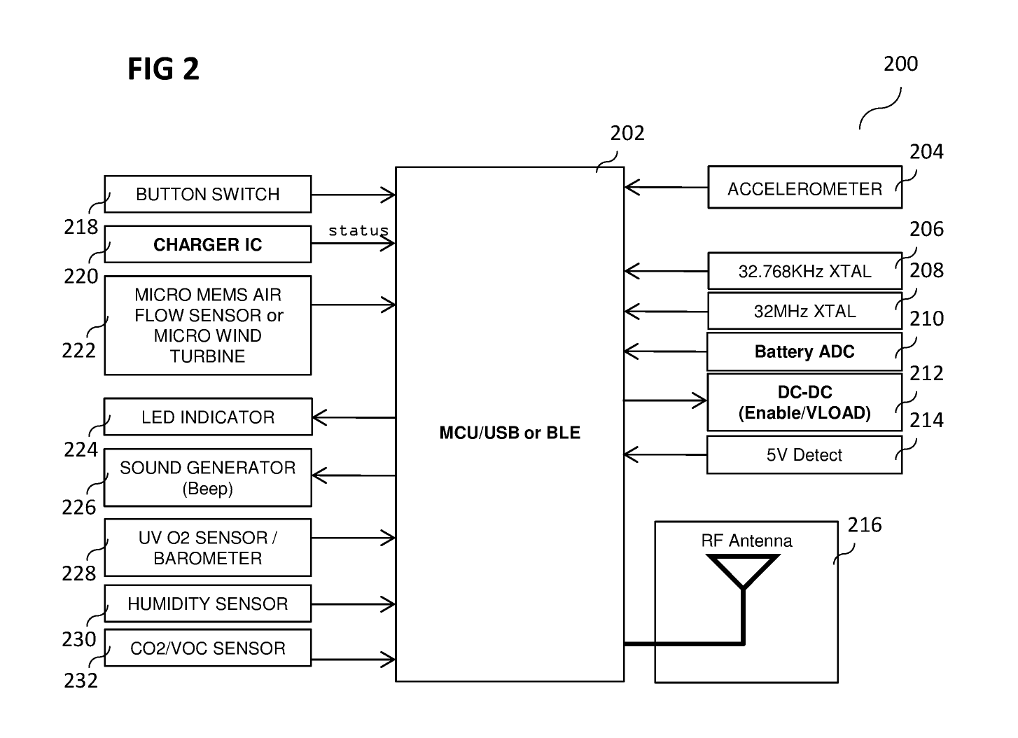

FIG. 2 shows a functional block diagram 200 of a breath measuring device according to various embodiments. A MCU (microcontroller unit) 202 may provide a USB communication interface and/or a BLE communication interface. An accelerometer 204, a 32.768 kHz XTAL (crystal) 206, a 32 MHz XTAL 208, a battery ADC (analog digital converter) 210, a DC (direct current)-DC circuit 212 (which may provide enable/VLOAD signals), a 5V detect circuit 214, an RF (radio frequency) antenna 216, a button switch 218, a charger IC (integrated circuit) 220, a micro MEMS air flow sensor (or a micro wind turbine) 222, and LED (light emitting diode) indicator 224, a sound generator 226 (which may be configured to generate a beep sound), an UV (ultraviolet) O2 sensor/barometer 228, a humidity sensor 230, and a CO2/VOC sensor 232 may be connected to the MCU 202. Besides measuring air flow speed, the micro MEMS air flow sensor (or the micro wind turbine) 222 may double off as a power generator.

Various embodiments may allow sports enthusiasts to track volume of breath intake which is linked to improved health and fitness levels, improved endurance levels and athletic performance, improved mental focus and brain health. Devices according to various embodiments may enable a user to know if he/she is breathing in the proper manner during exercise, and may recommend suggestions and advice to improve breathing techniques, or breathing patterns.

Portable devices according to various embodiments may allow enthusiast sports people to measure their breathing cycle and volume throughout their exercise regime, even outdoors. Measuring breathing patterns during training may help magnify player bad behaviors.

Devices according to various embodiments may be coupled with other external sensors for full spectrum of analysis. Such external sensors may for example be: A high speed camera, a heart rate sensor, and/or a thermometer. When the device according to various embodiments is coupled with other sensors, exertion and stress may be more easily corrected by coaches. When coupled with an oxygen sensor, a carbon dioxide sensor, a humidity sensor, a pressure sensor and/or a temperature sensor in the mask according to various embodiments, the fitness breath analyzer according to various embodiments may estimate the body's efficiency in processing oxygen. VOC (volatile organic compounds) sensors may alert the user if the environmental air is hazardous to the user.

Various embodiments may be used in wearable products.

According to various embodiments, low-cost equipment may be provided (in other words: expensive equipment may not be required).

The following examples pertain to further embodiments.

Example 1 is a sensor device comprising: an airflow sensor configured to generate a voltage based on an airflow; and an output circuit configured to output measurement data based on the voltage; wherein the airflow sensor is configured to provide the voltage as a power supply to the output circuit.

In example 2, the subject-matter of example 1 can optionally include a battery configured to power the airflow sensor and the output circuit.

In example 3, the subject-matter of any one of examples 1 to 2 can optionally include that the airflow sensor is configured to provide the voltage for charging the battery.

In example 4, the subject-matter of any one of examples 1 to 3 can optionally include that the airflow is breath.

In example 5, the subject-matter of any one of examples 1 to 4 can optionally include that the airflow sensor comprises at least one of a turbine or a microelectromechanical system.

In example 6, the subject-matter of any one of examples 1 to 5 can optionally include an oxygen sensor.

In example 7, the subject-matter of any one of examples 1 to 6 can optionally include a carbon dioxide sensor.

In example 8, the subject-matter of any one of examples 1 to 7 can optionally include a humidity sensor.

In example 9, the subject-matter of any one of examples 1 to 8 can optionally include a pressure sensor.

In example 10, the subject-matter of any one of examples 1 to 9 can optionally include a temperature sensor.

In example 11, the subject-matter of any one of examples 1 to 10 can optionally include an accelerometer.

In example 12, the subject-matter of any one of examples 1 to 11 can optionally include that the output circuit comprises an interface.

In example 13, the subject-matter of any one of examples 1 to 12 can optionally include that the interface is configured according to at least one of Bluetooth, Bluetooth low energy, and universal serial bus.

In example 14, the subject-matter of any one of examples 1 to 13 can optionally include that the sensor device is a sensor mask.

In example 15, the subject-matter of any one of examples 1 to 14 can optionally include a strap configured to attach the sensor device to a user's head.

Example 16 is a method for controlling a sensor device, the method comprising: controlling an airflow sensor to generate a voltage based on an airflow; controlling an output circuit to output measurement data based on the voltage; and controlling the airflow sensor to provide the voltage as a power supply to the output circuit.

In example 17, the subject-matter of example 16 can optionally include controlling a battery configured to power the airflow sensor and the output circuit.

In example 18, the subject-matter of any one of examples 16 to 17 can optionally include that the airflow sensor is controlled to provide the voltage for charging the battery.

In example 19, the subject-matter of any one of examples 16 to 18 can optionally include that the airflow is breath.

In example 20, the subject-matter of any one of examples 16 to 19 can optionally include that the airflow sensor comprises at least one of a turbine or a microelectromechanical system.

In example 21, the subject-matter of any one of examples 16 to 20 can optionally include controlling an oxygen sensor.

In example 22, the subject-matter of any one of examples 16 to 21 can optionally include controlling a carbon dioxide sensor.

In example 23, the subject-matter of any one of examples 16 to 22 can optionally include controlling a humidity sensor.

In example 24, the subject-matter of any one of examples 16 to 23 can optionally include controlling a pressure sensor.

In example 25, the subject-matter of any one of examples 16 to 24 can optionally include controlling a temperature sensor.

In example 26, the subject-matter of any one of examples 16 to 25 can optionally include controlling an accelerometer.

In example 27, the subject-matter of any one of examples 16 to 26 can optionally include that the output circuit comprises an interface.

In example 28, the subject-matter of any one of examples 16 to 27 can optionally include that the interface is configured according to at least one of Bluetooth, Bluetooth low energy, and universal serial bus.

In example 29, the subject-matter of any one of examples 16 to 28 can optionally include that the sensor device is a sensor mask.

In example 30, the subject-matter of any one of examples 16 to 29 can optionally include that the sensor device comprises a strap configured to attach the sensor device to a user's head.

While the invention has been particularly shown and described with reference to specific embodiments, it should be understood by those skilled in the art that various changes in form and detail may be made therein without departing from the spirit and scope of the invention as defined by the appended claims. The scope of the invention is thus indicated by the appended claims and all changes which come within the meaning and range of equivalency of the claims are therefore intended to be embraced.

1. A sensor device comprising:
an airflow sensor configured to generate a voltage based on an airflow; and
an output circuit configured to output measurement data based on the voltage;
wherein the airflow sensor is configured to provide the voltage as a power supply to the output circuit.

2. The sensor device of claim 1, further comprising:
a battery configured to power the airflow sensor and the output circuit.

3. The sensor device of claim 2,
wherein the airflow sensor is configured to provide the voltage for charging the battery.

4. The sensor device of claim 1,
wherein the airflow is breath.

5. The sensor device of claim 1,
wherein the airflow sensor comprises at least one of a turbine or a microelectromechanical system.

6. The sensor device of claim 1, further comprising:
at least one of an oxygen sensor, a carbon dioxide sensor, a humidity sensor, a pressure sensor, a temperature sensor, and an accelerometer.

7-11. (canceled)

12. The sensor device of claim 1,
wherein the output circuit comprises an interface.

13. The sensor device of claim 12,
wherein the interface is configured according to at least one of Bluetooth, Bluetooth low energy, and universal serial bus.

14. The sensor device of claim 1,
wherein the sensor device is a sensor mask.

15. The sensor device of claim 1, further comprising:
a strap configured to attach the sensor device to a user's head.

16. A method for controlling a sensor device, the method comprising:
controlling an airflow sensor to generate a voltage based on an airflow;
controlling an output circuit to output measurement data based on the voltage; and
controlling the airflow sensor to provide the voltage as a power supply to the output circuit.

17. The method of claim 16, further comprising:
controlling a battery configured to power the airflow sensor and the output circuit.

18. The method of claim 17,
wherein the airflow sensor is controlled to provide the voltage for charging the battery.

19. The method of claim 16,
wherein the airflow is breath.

20. The method of claim 16,
wherein the airflow sensor comprises at least one of a turbine or a microelectromechanical system.

21. The method of claim 16, further comprising:
controlling at least one of an oxygen sensor, a carbon dioxide sensor, a humidity sensor, a pressure sensor, a temperature sensor, and an accelerometer.

22-26. (canceled)

27. The method of claim 16,
wherein the output circuit comprises an interface.

28. The method of claim 27,
wherein the interface is configured according to at least one of Bluetooth, Bluetooth low energy, and universal serial bus.

29. The method of claim 16,
wherein the sensor device is a sensor mask.

30. The method of claim 16,
wherein the sensor device comprises a strap configured to attach the sensor device to a user's head.
